(19) United States
(12) Patent Application Publication
(10) Pub. No.: US 2024/0345099 A1
(43) Pub. Date: Oct. 17, 2024

(54) METHOD AND DEVICE FOR ABSOLUTE QUANTIFICATION OF ANALYTES

(71) Applicant: Cenios GmbH, Bottrop (DE)

(72) Inventors: Mohammad SALEHI, Bottrop (DE); Angela HAMANN-STEINMEIER, Osnabrück (DE)

(21) Appl. No.: 18/683,332

(22) PCT Filed: Aug. 11, 2022
(86) PCT No.: PCT/EP2022/072560

§ 371 Date: Feb. 13, 2024

(30) Foreign Application Priority Data
Aug. 13, 2021 (DE) 10 2021 121 118.2

(51) Int. Cl. G01N 33/68; B82Y 5/00; B82Y 15/00; G01N 21/64; G01N 21/65; G01N 33/58

(52) U.S. Cl. CPC G01N 33/6851; B82Y 5/00; B82Y 15/00; G01N 21/6428; G01N 21/658; G01N 33/587; G01N 2021/6439

(57) ABSTRACT

The invention relates to a method for the absolute quantification of at least one analyte by means of a rapid test, in particular by spectroscopic determination. In order to provide a method and a device for the absolute quantification of analytes and to enable the creation of a database for the improved individual dosing of therapeutics, whereby a particularly accurate quantification of at least one analyte is possible in a simple manner, particularly inexpensively, particularly quickly and at any location without the need for a laboratory, it is provided, that at least one nanomaterial is used as a luminescent substance for quantification, which interacts directly or indirectly with the analyte, wherein at least one signal generated by the nanomaterial is measured and wherein in addition an internal and/or external standard series, in particular by means of Raman-labeled targets and/or fluorescence-labeled targets, is measured and subsequently the generated signal is standardized or referenced on the basis of the internal and/or external standard series, so that an absolute quantification is achieved.

The invention relates to new methods for the absolute quantification of analytes, in particular in rapid tests and/or on membranes. The measured values obtained can be used to adjust or personalize the dose of medication and/or to create a digital database. A database can be used to personalize the dosage of medication for a patient without time-consuming and complicated methods.

Rapid test methods or POCT (point of care test) are often lateral flow assays (LFA) or vertical flow assays (VFA), also known as test strips. These products are inexpensive to produce and easy to store, especially between 2° C. to 30° C. They then provide reliable “on-site” results outside and inside a laboratory, typically within 3-15 minutes. The best-known example of this is various COVID-19 or pregnancy tests, which can be used by specialists or at home by laypersons and easily analyzed. These diagnostics are referred to as conventional tests, which only provide a positive or negative result that cannot be scaled or quantified. In scientific jargon, such results are referred to as qualitative results (yes/no or positive/negative), which are preferably visible to the naked eye.

In the case of pregnancy or an infectious disease, such as COVID-19, a qualitative result is usually sufficient. However, such qualitative results are not sufficient for many sought-after substances, such as medications and toxins. If a test is positive, the experts want to know what quantity of the substances or analyte they are looking for is present. Accordingly, quantitative measurements are preferred. The measured quantity can be a medication level or a concentration of an analyte, e.g. cytostatics or antibiotics, particularly in the blood of patients, or antibiotic and toxin contamination, for example in food.

The former quantifications are already known as therapeutic drug monitoring (TDM) and are carried out using methods such as high-performance liquid chromatography (HPLC) or HPLC with mass spectrometry coupling (HPLC-MS). Therapeutic drug monitoring can be used, for example, to reduce side effects as well as medication consumption and healing times in humans and animals.

For the latter application, a measurement of food contamination, quality control could be carried out during and after the production of a food product. Currently, POCT methods can only provide qualitative or semi-qualitative results. Thus, they cannot provide a reliable quantification of the target analytes.

In contrast to POCT methods, quantitative methods such as HPLC, HPLC-MS or ELISA (Enzyme-Linked Immuno-Sorbent Assay) can guarantee reliable quantification. Other methods such as immunoassays, chemiluminescence immunoassays, fluorescence immunoassays, colorimetric immunoassays and radio immunoassays can also be used for therapeutic drug monitoring. Like ELISA methods, these methods are based on antibody detection.

The quantitative methods are established as so-called gold standards. The main difference between quantitative and qualitative methods is the existence of the standard series. Due to the lack of standard series, POCT methods can only provide qualitative or semi-qualitative readings. The use of metallic nanomaterials as reagents can be seen as another difference between POCT and above mentioned methods.

The corresponding conventional state-of-the-art readers work either with a camera or corresponding scanning systems. The colored test and control bands are detected by the CCD or CMOS camera using optical density (OD). Scanning methods are mostly used in signal acquisition by the colorimetric, reflectrometric and fluorescence-based techniques. LFA readout methods using fluorescent dyes instead of gold nanoparticles have also been developed and marketed. The fluorescence-based method works with a fluorescence sensor that can detect the fluorescently labeled TL and CL. With such quantification methods, only a constant CL value exists as a reference. Similarly, the development or improvement of mobile Raman readout devices should not be regarded as a novelty. Such device was used by the FBI in 2001. Although readout devices available on the market are easy to handle and inexpensive, they do not provide accurate results without a standard series.

Apart from low sensitivity and precision, all prior art methods require the test strips to be completely dry, which takes additional time and significantly increases the time between sampling and measurement. The measurement results in the wet state differ from the dry state, as the optical density is different in the wet and dry state, which can generate false results. The falsification of the results is also to be expected in the case of reflectrometric Raman and/or fluorescence-labeled measurements, whereby the signals (light, Raman and fluorescence) from nanomaterials are reflected or recorded by the detector.

The invention is therefore based on the object of providing a method and a device for the absolute quantification of analytes as well as enabling the creation of a database for improved individual dosing of therapeutic agents, whereby a particularly accurate quantification of at least one analyte is possible in a simple manner, particularly inexpensively, particularly quickly and at any location without the need for a laboratory.

According to the invention, the object is solved by a method according to claim 1, a device according to claim 14, the use according to claim 15 and a method according to claim 16. Advantageous further embodiments are given in the dependent claims and in the description below.

In the method according to the invention for the absolute quantification of at least one analyte by means of a rapid test, in particular by spectroscopic determination, at least one nanomaterial is used as a luminescent substance, preferably raman-active materials and in particular IgG-coupled raman-active nanomaterials, for quantification, wherein this material interacts directly or indirectly with the analyte, wherein at least one signal generated by the nanomaterial is measured and wherein in addition an internal and/or external standard series, in particular by means of Raman-labeled targets and/or fluorescence-labeled targets, is measured and subsequently the generated signal is standardized or referenced on the basis of the internal and/or the external standard series, so that an absolute quantification of the analyte is achieved.

Furthermore, the invention relates to a device for the absolute quantification of at least one analyte by means of a rapid test, in particular by spectroscopic determination and preferably by surface-enhanced Raman scattering, with at least one nanomaterial as luminescent substance for quantification, which interacts indirectly or directly with the analyte, wherein the rapid test is provided, to be measured by means of a spectrometer or by means of at least one filter and at least one sensor in order to measure at least one signal generated by the nanomaterial as well as an internal and/or external standard series, in particular by means of Raman-labeled targets and/or fluorescence-labeled targets, wherein the concentration of the analyte being standardized or referenced by means of a data processing device using the internal and/or the external standard series, so that an absolute quantification of the analyte is achieved.

Furthermore, the invention relates to a use of the method according to the invention and/or the device according to the invention in human medicine, in veterinary medicine and/or in the food industry, in particular in order to carry out a quantitative determination of active substances, medicaments and/or toxins by means of a rapid test.

Finally, the invention also comprises a method for creating a database for improved individual dosing of therapeutics, wherein absolutely quantified measured values obtained by means of the method according to the invention and/or further measured data and/or parameters of the patient are used to make an adjustment or personalization of the medication dose and/or to make dosing recommendations for medications or active ingredients for future patients by means of AI algorithms and/or by means of big data approaches.

A quantification method, in particular based on LFA and/or Raman-active nanomaterials, called qSERS, has been developed for the absolute quantification of the analytes. To quantify the analytes, the noble metal nanoparticles are preferably coated with a self-assembled single layer or a monolayer of organic molecules, the Raman markers, which emit the characteristic scattered light “SERS” (Surface Enhanced Raman Scattering). By coupling such nanomaterials to ligands, e.g. antibodies, SERS nanotags can be produced. In the qSERS method, the nanomaterials are preferentially exchanged from the LFA strips with SERS tags, which enable quantification by the characteristic signal of the marker bound to the antibody (FIG. 1). A SERS readout device is preferably used to evaluate the Raman and SERS signals. The readout device has particularly preferred measurement times of 5-15 seconds, making it the fastest and most accurate LFA readout device in the world.

A central aspect of the present invention is to create or measure an internal standard series in order to then enable absolute quantification of the analyte. The standardization of the present invention also makes it possible to detect and correct any signal losses.

A rapid test within the meaning of the invention is a test that can be carried out with little time and/or equipment and, in particular, preferably allows an analyte to be quantified directly. The rapid test is preferably evaluated spectroscopically, with one or more spectroscopy techniques being used simultaneously or successively for this purpose. It is particularly preferred that the evaluation of the rapid test or the measurement is carried out exclusively spectroscopically. Raman spectroscopy and/or UV, UV-VIS, and/or fluorescence and/or IR spectroscopy are also preferably used for this purpose. Alternatively, only a filter and a sensor can be used for evaluation.

According to the invention, at least one nanomaterial is used as a luminescent substance to quantify the analyte, whereby the use of several different nanomaterials is also conceivable. In this context, a nanomaterial as a luminescent substance is understood to be any luminescent substance which is so small that it can interact directly with the analyte and is preferably at most 200 micrometers in size and particularly preferably at most 1 nanometer in size. The luminescent substance has the task of interacting with the analyte and at the same time being spectroscopically detectable.

With an internal and/or external standard series first of all basically a possibility for quantification is created, wherein a standard series preferably comprising at least two, particularly preferably at least three and very particularly preferably at least five values. In an internal standard series, for example, a known amount of an analyte or a reference substance can be present, which is then preferably measured at different wavelengths and/or irradiation durations and/or exposure durations, in particular durations of a laser pulse. In the case of an external standard series, a series of different concentrations of an analyte or a reference substance is preferably kept ready.

Although the nanomaterial as a luminescent substance can be measured by any method and can be adjusted accordingly to any light source and/or wavelength, an advantageous further development of the method according to the invention provides that at least one nanomaterial as a luminescent substance is a Raman-active material, in particular an IgG-coupled Raman-active nanomaterial, and/or the measurement of the signal generated by the nanomaterial is carried out by means of surface-enhanced Raman scattering, so that the generated signal is a surface-enhanced Raman scattering signal, i.e. a SERS signal.

In order to be able to quantify the SERS signals in the measurement range of the standard series, the SERS signal measurements are preferably carried out with lower laser power. In the same way, absolute quantification can be determined with VLA. The use of VLA leads to an advantageous minimization of the hook effect (high-dose hook effect), which is usually the cause of a false-low quantification of analytes.

A preferred embodiment of the method according to the invention for the absolute quantification of at least one analyte provides that a background material, a component, a plastic part, a plastic housing and/or a nitrocellulose membrane of the rapid test is used as the target to be measured for measuring an internal standard series, by means of which a constant measurement signal can be measured for defined measurement parameters independently of the concentration of the analyte and thus this background measurement signal can be used for standardization of the generated signal of the nanomaterial.

Furthermore, it is preferred that a control line or a control position of the rapid test is measured to measure an internal standard series and the measured value obtained is used as a reference value for the standardization of the generated signal of the nanomaterial, whereby a supplementary calculation of the analyte is preferably carried out using a 4-parameter logistic.

According to a preferred further development of the method according to the invention for absolute quantification, the measurement of the internal and/or the external standard series is carried out by means of Raman-labeled targets and/or by means of fluorescence-labeled targets. Accordingly, it is preferred that the internal and/or the external standard series is measured by means of Raman spectroscopy and/or by means of fluorescence spectroscopy. It is particularly preferred that the at least one target is measured with different intensities of an irradiated light, with different laser powers and/or with a changed laser focus in order to measure an internal standard series, so that several measurements of a standard series are obtained.

Internal standard series can be created additionally or exclusively by changing the laser focus, especially since the maximum Raman signals can only be expected at the optimum focus. For example, the laser probe can move away from the optimum plane in 6 steps (along a Z-axis) and perform a Raman measurement in a new position each time. For better understanding, standard series with 5-7 points were intentionally generated and shown in the embodiment examples given as well as in the figures. However, the number of points can optionally be changed, whereby any number of measuring points is conceivable. A number between 3 and 15 measuring points is preferred, particularly preferably between 4 and 10 measuring points and especially preferred are between 5 and 7 measuring points, as more measuring points increase the measuring effort and the measuring time and fewer measuring points reduce the reliability of the measuring result.

Apart from the methods mentioned above, however, an alternative or additional method for quantification without a plastic housing and/or without a nitrocellulose membrane, in particular of an LFA strip without a plastic housing, is possible. Separately-prepared signal-active and preferably metallic nanomaterials can be used to generate a standard series for POCT methods.

A preferred embodiment of the method according to the invention provides that several measuring surfaces, each with a different concentration of a separately prepared signal-active material and/or at least one nanomaterial, preferably metallic nanomaterials, are provided for a measurement of an external standard series, wherein the signal-active material and/or the nanomaterial is particularly preferably immobilized on an attachment or on the rapid test and in particular preferably also sealed.

In this case, the signal-active nanomaterials are particularly preferably prepared in different concentrations, immobilized on an attachment and, in particular, preferably also sealed. The sealing serves to ensure the durability and multiple use of the signal-active nanomaterials of the attachment, since the attachment, in contrast to the test strips, is preferably not a disposable product and is intended to be used several times. The signal-active nanomaterials can, for example, be formed in the form of a barcode as an attachment. However, the shape and placement of the individual reference materials, in particular the signal-active nanomaterials, is immaterial and can be made as desired, for example in the form of a row and/or an array of strips, dots and/or areas. The signal-active nanomaterials refer to all nanocrystalline luminescent substances, in particular Raman-active quantum dots upconversion.

With all measurement methods, apart from measuring the plastic housing, a spectrometer can be dispensed with alternatively or additionally and optical filters and sensors can be used instead of the spectrometer. This allows Raman and/or fluorescence measurements in particular to be carried out.

Apart from POCT, an advantageous further development of the method according to the invention can also be used in a Western blot analysis for the purpose of quantifying the target molecules. In general, an embodiment of the method according to the invention in which the rapid test is a lateral flow assay or a vertical flow assay or Western blot is preferred. The target proteins on the membrane in the Western blot method are labeled with an antibody, abbreviated as IgG, for example. The immobilized antibodies, or IgGs, are then made visible to the naked eye with the chemicals or the protein A-coupled nanomaterials using a camera. Visualization and semi-quantitative evaluation is carried out using suitable chemicals and the chemiluminescence technique, for example. In a further development of the method, ligands such as the Haevy chain of IgGs and/or streptavidin are transferred to a substrate such as an SDS gel as reference proteins. After immunodetection, the membranes are preferably treated with ligand-bound nanomaterials. The nanomaterials bind to specific IgGs that have recognized their targets, together with one or both reference proteins of known concentration. Thus, the unknown concentrations can be determined using the signals of the standard series and the reference protein of known concentration.

In a generally preferred embodiment of the method according to the invention, quantum dots upconversion and/or nanomaterials are used for quantification of the analyte, whereby the nanomaterials and in particular precious metal nanoparticles are preferably coated with a self-assembling single layer or a monolayer of organic molecules as Raman markers and/or such materials or nanomaterials are coupled to ligands such as protein A, protein G, protein A/G, protein L, biotinylated antibodies or enzyme-coupled and fluorescent antibodies for quantification of the analyte.

In general, gold, silver, alloyed gold and/or silver, gold nanostars, Raman-active nanomaterials, quantum dots and upconversions can be used as nanomaterials. These nanomaterials can be coated with any ligands, such as protein A, protein G, protein A/G, protein L, biotinylated antibodies or enzyme-coupled and/or fluorescent antibodies. For example, the nanomaterial may comprise one or more of the luminescent substances and in particular as nanocrystalline luminescent substances LaPO4:Ce3+, LaPO4:Pr3+, LuPO4:Pr3+, LaPO4:Tm3+LuPO4:Dy3+, LuPO4:EU3+, LuPO4:Tb3+, LuPO4:Tm3+ and particularly preferably formed therefrom. The nanomaterials or microparticles produced by doping can also be formed from a variety of plastic polymers, such as polystyrene, polyacrylamides, polymethyl methacrylate, etc. There are no limits to the choice of nano- and microparticles for solving the luminescing task in this process. In principle, any nanocrystalline luminescent substance or luminescent microparticles or plastic polymers can be used in the process according to the invention.

In general, the methods can be used to quantify the analytes with internal and external standard series for reflectometric Raman and fluorescence-labeled quantifications.

In addition, one aspect of the present invention relates to the possibility of combining the various signaling substances and in particular several nanomaterials as luminescent substances with each other. Some signaling substances, such as fluorescent dyes, suffer from photobleaching and lose their fluorescence ability due to multiple irradiation of the fluorophore with the excitation light or even during storage. Such unstable fluorophores can be coupled to any ligand, such as biomolecules, for quantification. However, such unstable fluorophores lose their signaling when they are used as barcodes to create the standard series. In contrast, nanocrystalline luminescent substances such as quantum dots, upconversion and other nanocrystalline materials hardly lose their signal stability over time and/or with increasing exposure time. By specifically selecting the appropriate nanocrystalline luminescent substances, in particular as barcodes, unstable fluorescent dyes can be used, in particular for making the strips, as the strips are generally only exposed once to the excitation light. In one possible embodiment of the method according to the invention, at least one nanocrystalline luminescent substance is used to measure the standard series and/or at least one fluorescent dye is used as a luminescent substance to quantify the analyte.

Currently, the wavelengths of approx. 360 nm (±10 nm) are commonly used as a light source to generate signals at approx. 610 nm (±10 nm). Preferably, the standard series and a test line and/or a control line are exposed simultaneously, with exposure at a wavelength of approx. 360 nm (±10 nm) and measurement of the emitted signals at approx. 610 nm (±10 nm) being particularly preferred. In addition, the exposure can also take place non-simultaneously and, in particular, immediately one after the other or sequentially. In general, there are also no limits to the use of other wavelengths as a light source or for sensors for signal perception. Therefore, simultaneous exposure of the various nanomaterials or luminescent substances and, in particular, of test and/or control lines and/or even a standard series in all wavelengths is also possible by means of the method according to the invention. It is also possible to measure the emitted signals at any wavelength, with a wavelength range from UV to IR being preferred.

Furthermore, according to an advantageous embodiment of the method according to the invention, it is preferred that a measurement and in particular a spectroscopic examination of a positive and a negative control is carried out in parallel with the measurement of the signal of the nanocrystalline luminescent substance generated by the nanomaterial. The rapid test of the present invention is preferably designed as a multiple cassette. In particular, the negative and positive control can preferably be examined in parallel with the test sample or samples. In the negative control or negative reference, the substance sought is missing, which means that in an intact test and the determination of the negative reference only one control line may interact with the reagents. The appearance of a band on the test position then indicates contamination of the negative reference or faulty production of the test.

It is also preferable to determine a positive control or a positive reference. For this purpose, a positive control or a positive reference is used that contains the substances sought in a defined quantity. As a result, at least one band is formed on the test strip as a reference. During the production of the positive control strips, capture ligands are immobilized on a carrier material, preferably in different concentrations. This allows more than one line to be formed by applying the positive control and, accordingly, further additional measured values can be generated in this way, apart from the barcode signals as a standard series.

A further important aspect of the invention relates to the use of big data approaches and/or artificial intelligence to enable improved individualized dosing of therapeutics. In particular, this preferably relates to the creation of a corresponding database. In addition, it is particularly preferred that quantitative data from the method according to the invention is used for this purpose.

Currently, humans and animals are treated with the medications based on existing protocols and/or characteristics, such as age, weight and gender. By means of the present invention, the degree of individual metabolization can be determined “on site”. In humans (and animals) of the same sex, age and weight, body functions such as kidney and liver function may be different. Currently, there are no quantitative POCT kits that can solve these tasks and enable TDM. However, the digitization of data for the treatment of humans and animals offers numerous advantages. When securing personal data, information such as age, gender, origin, previous illnesses and vital functions can be stored anonymously in the form of a barcode.

HPLC and ELISA can be used as the gold standard for determining the measuring ranges and defining the interference factors. Parallel measurements, such as HPLC and ELISA, are an indispensable prerequisite for the certification of quantitative POCT products. Corresponding databases with such data can be used to confirm the therapeutic areas, for example for cytostatics and antibiotics. This should generate data that can be used to make more precise dose adjustments for the substances. By analyzing the stored or historical data, a better dose recommendation for the substances should be possible. It should be noted that the decision remains with the treating physician. By storing the data, algorithms from the field of data analytics and machine learning can also be used.

By storing the data, big data approaches can also be pursued. For the purpose of individual dosing of the medication, certification of the POCT products and creation of the big data or database, individual TDM examinations can be carried out for each person. These TDM examinations are carried out using the method according to the invention and/or using gold standard methods. These measurements can be carried out before, during and/or after medical treatments for the purpose of individualized dosage recommendations. The resulting dosages can also be stored in the form of a barcode for the respective patient.

With the help of population pharmacokinetic analyses, digitalization, use of the data obtained and/or artificial intelligence, the possible influencing factors on the kinetics can be identified and quantified. The larger and more comprehensive the personal data collected, the smaller the deviations in medication dosage.

Ideally, artificial intelligence (AI) in the form of software or an app can also enable individualized dosage recommendations based on the stored personalized data without TDM measurements. The stored data and the clinical knowledge gained from it preferably serve as a memory for the artificial intelligence. The artificial intelligence can recommend a precise dosage to physicians based on the stored information.

In fact, AI can make better decisions about medication dosage based on stored personal data and routine laboratory results than conventional protocols. Since the AI has access to countless determined personal dosages, the AI can decide on the optimal dosage better than the respective individual physician. In addition, people in rural areas or third world countries with suitable big data and without real TDM measurements can take advantage of personalized medication dosing.

Previous data show that the quantitative POCT method according to the invention with urine and blood or plasma samples shows an excellent correlation with the gold standard methods. In particular, the measurements of cytostatics and antibiotics in urine of humans and animals are advantageous, as the sampling is not invasive and substances remain stable for longer in comparison.

The anonymous digitization of patient data opens up enormous opportunities for therapy optimization. However, this digitization has hardly been researched or used in the field of TDM.

Further details of the invention are shown in the drawings. The figures show:

FIG. 1: A schematic representation of a dilution series in SERS-LFA (A). Representation of the Raman background signal (o) and corresponding SERS signals of the test strips (B). The signals of the control lines are not shown.

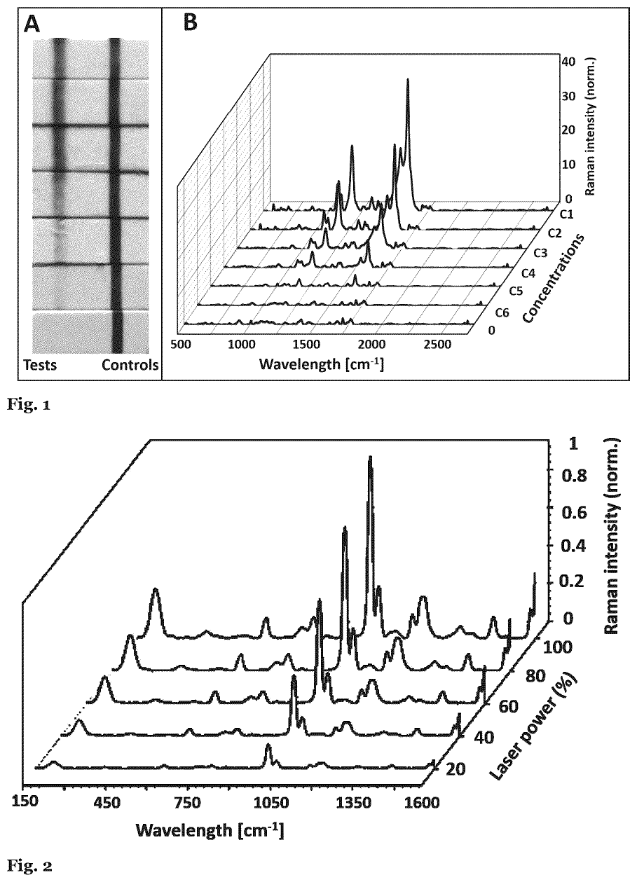

FIG. 2: Generation of the internal standard series by changing the laser power on the plastic housing of the kit. The qSERS method allows a reliable standard series to be generated with stable internal Raman signals.

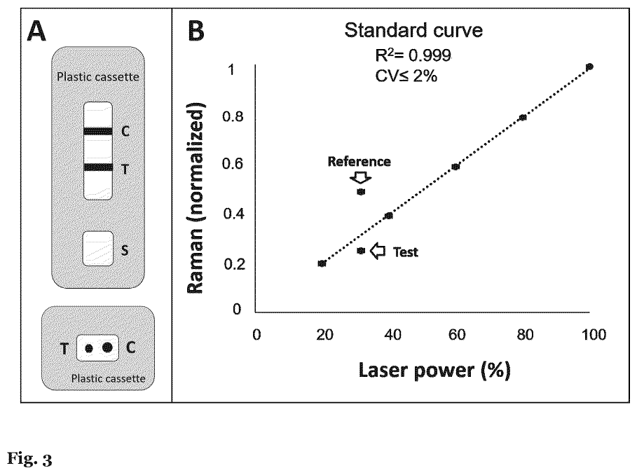

FIG. 3: Schematic representation of the LFA (A top) and VLA (A bottom) in the plastic housings. To create the internal standard series, the signal measurements of the plastic housings were recorded with different laser powers (FIG. 2). By determining and standardizing the signal area of the plastic housings, a stable internal standard series with a reliable correlation coefficient could be created. The SERS measurements of the control and test lines were carried out with low laser power.

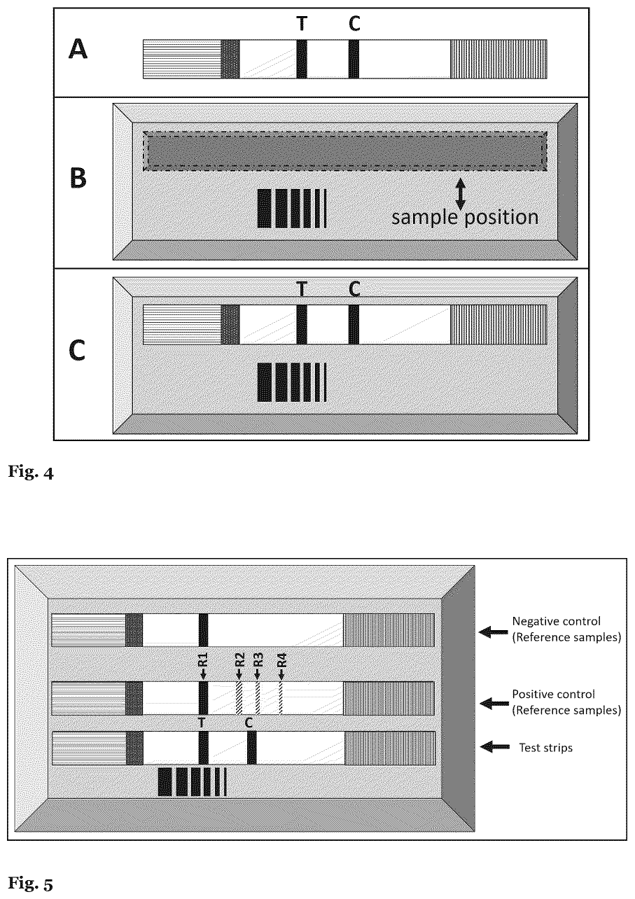

FIG. 4: Schematic representation of the LFA (A). The LFA strips are placed in an attachment with a well (B). The signals of the nanomaterials and their relevant signal-active standard series (barcode) can be detected simultaneously or non-simultaneously by a readout device (C).

FIG. 5: Schematic representation of the LFA with a separate negative and positive control as reference samples parallel to the test sample.

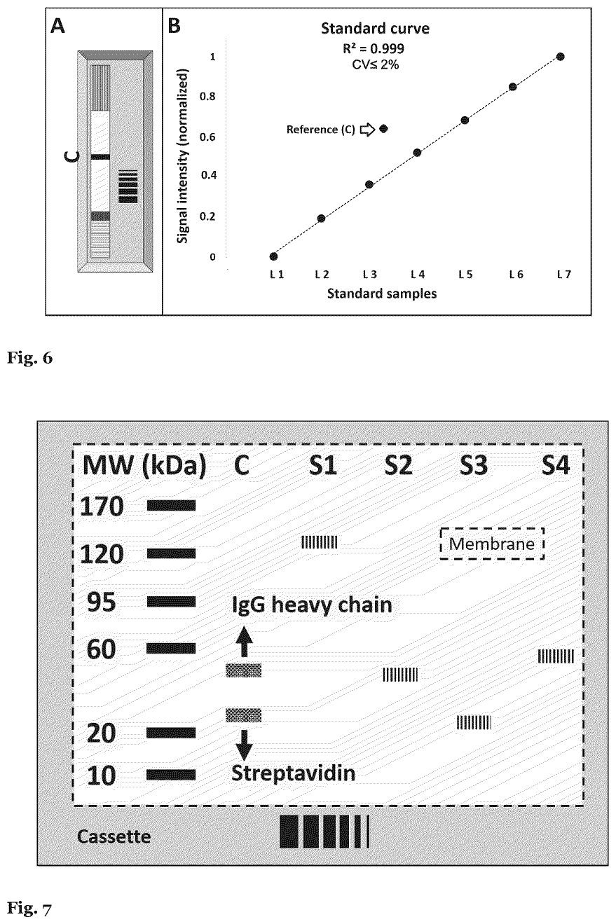

FIG. 6: Schematic representation of the LFA with a control line (reference), (A). By standardization of the generated signals from the strips (barcode), a reproducible standard series can be generated, whereby the maximum signal is shown as 1 (at L7) and minimum signal as 0 (plastic).

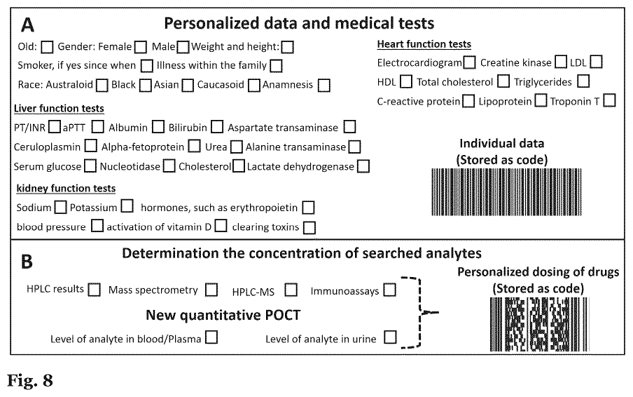

FIG. 7: Visualization of the target proteins in the Western blot. The size of the target protein can be determined using various molecular weight markers (10-170 kDa) (left). The ligands, such as the Haevy-chain of the IgGs and streptavidin, can be used in certain concentrations as reference proteins (sample C) for the quantification of other target molecules (S1-S4).

FIG. 8: Information such as age, gender, origin, previous illnesses and vital functions are stored anonymously in the form of a barcode (A). The individual dosage of medication is determined on the basis of the TDM examinations and stored in the form of another barcode (B).

A first version of a rapid test procedure, also known as a POCT (point of care test), is formed either as lateral flow assays (LFA) or vertical flow assays (VFA) and is also referred to as a test strip. To perform a test, a solution containing an analyte to be quantified is placed on the test strip.

In this embodiment of the method according to the invention, a readout device then simultaneously records several signals during the quantification of the LFA or VFA strips. Some of these signals can be generated by background materials, e.g. from a nitrocellulose membrane of the LFA strips or from a plastic housing. The readout device performs at least Raman spectroscopic measurements. In particular, Raman background signals of the plastics of the housing and especially preferably of the nitrocellulose membrane are separated and/or used together with the plastic housing for standardizations of the signals. The at least second measured signal is a SERS signal, i.e. a signal of Surface-Enhanced Raman Scattering, which is generated by nanomaterials located on the rapid test and serving as luminescent substances (see FIG. 1).

While the Raman signals of the backgrounds, i.e. the plastic, the plastic housing and/or the nitrocellulose membrane, for example, are constant signals, the SERS signals show different signal intensities depending on the concentration of the analytes. The Raman signals of the background are used in qSERS according to the present invention for standardization of the SERS signals of the immune complex (see FIG. 1).

The respective measurement conditions, such as the laser power, the exposure time and the exact positioning on the focal plane, can change the SERS signals and cause large signal deviations. By standardizing the concentration-dependent SERS signals to the constant Raman signals, such deviations can be reduced or even quasi eliminated. Another advantage of the qSERS method according to the invention is the possibility of creating the necessary internal standard series using the plastic housing of the rapid test, especially with a change in laser power (see FIG. 2). A stable internal standard curve with a reliable correlation coefficient (R²=0.999, CV≤2%) can be generated by calculating and standardizing the signal area, in particular the wavenumbers 967-1047 cm⁻¹, of different spectra (see FIG. 3).

It is also possible to use one or more control lines of the rapid test according to the invention, in particular for each strip, as a reference value. The existence of a control line can be used for an additional calculation of the analyte via a 4-parameter logistic (4 PL), which, in addition to the internal standard series, opens up the possibility of a double control. As a rule, the control lines provide stable and reliable SERS signals. Therefore, the SERS signals of the control line can be used as a reference value in addition to the internal Raman standard curve for evaluation (see FIG. 3). With the double control described, quantification by qSERS can be more accurate than with the conventional HPLC or ELISA technique. As an example, the SERS measurements in FIG. 3 were determined by normalization or referencing to the Raman signals of the nitrocellulose membrane and the plastic housing at LFA.

Apart from the methods mentioned above, however, an alternative or additional method for quantification without a plastic housing and/or without a nitrocellulose membrane, in particular of an LFA strip without a plastic housing, is possible, as shown in FIG. 4A.

In this case, the signal-active nanomaterials are particularly preferably prepared in different concentrations and immobilized on an attachment and preferably also sealed (see FIG. 4B). The sealing serves to ensure the durability and multiple use of the signal-active nanomaterials of an attachment, since the attachment, in contrast to the test strips, is preferably not a disposable product and is to be used several times. FIG. 4B shows an example of the various signal-active nanomaterials in the form of a barcode as an attachment. However, the shape and placement of the individual reference materials, in particular the signal-active nanomaterials, is immaterial and can be made as desired, for example in the form of a row and/or an array of strips, dots and/or areas.

FIG. 4C shows an embodiment of a rapid test for simultaneous and/or non-simultaneous scanning of the standard series and the sample by a readout device. By standardizing the generated signals from the strips or the barcode with different and/or differently concentrated signal-active nanomaterials, a reproducible standard series can be generated, whereby the maximum signal is particularly preferably displayed as 1 after standardization and/or the minimum signal is also preferably displayed as 0 after standardization. In addition to this, a control line can also be used as a reference for an additional calculation of the analyte via a 4-parameter logistic (4 PL), as shown in FIGS. 3 and 6. The shape and placement of the signal-active nanomaterials as standard samples can be changed as desired. In this embodiment, the signal-active nanomaterials are all nanocrystalline luminescent substances, in particular raman-active quantum dots upconversion.

One way to generate a standard series for rapid test (POCT) procedures is to prepare the signal-active nanomaterials in various concentrations, immobilize them on a attachment and, in particular, preferably also seal them (see FIG. 4B). The sealing serves to ensure the durability and multiple use of the signal-active nanomaterials of the attachment, as the attachment, in contrast to the test strips, is preferably not disposable and is intended to be used several times. FIG. 4B shows an example of the various signal-active nanomaterials in the form of a barcode as an attachment. However, the shape and placement of the individual reference materials, in particular the signal-active nanomaterials, is immaterial and can be made as desired, for example in the form of a row and/or an array of strips, dots and/or areas.

FIG. 4C shows the possibility of optional simultaneous and/or non-simultaneous scanning of the standard series and the sample by a readout device. By standardizing the generated signals from the strips or the barcode, a reproducible standard series can be generated, whereby the maximum signal is particularly preferably displayed as 1 after standardization and/or the minimum signal is also preferably displayed as 0 after standardization. In addition to this, a control line (reference) can also be used for an additional calculation of the analyte via a 4-parameter logistic (4 PL), as shown in FIGS. 3 and 6. The shape and placement of the signal-active nanomaterials as standard samples can be changed as desired.

According to various European guidelines, diagnostic procedures must be able to determine additional values, such as a known positive and a negative control serum, in parallel with the tested samples. For the parallel qualitative determination of several substances, various multiple cassettes have already been developed and commercialized, especially for multidrug tests. However, this technology has not yet been used for the absolute quantification of substances, as in the present invention. Accordingly, the rapid test of the present invention can also be designed as a multiple cassette in order to comply with the relevant European regulations. The negative and positive controls can preferably be tested in parallel with the test sample(s). In the negative control or negative reference, the substance being sought is missing, which means that only one control line may interact with the reagents if the test is intact and the negative reference is determined. The appearance of a band on the test position then indicates contamination of the negative reference or faulty production of the test.

It is also preferable to determine a positive control or a positive reference. For this purpose, a positive control or a positive reference is used, which contains the substances sought in a defined quantity. As a result, at least one band is formed on the test strip as a reference, as shown as R1 in the center of FIG. 5. When producing the positive control strips, capture ligands can optionally be immobilized on the carrier material in different concentrations. This allows more than one line to be formed by applying the positive control, as shown as R2, R3 and R4 in the center of FIG. 5. Accordingly, apart from the barcode signals as a standard series, further additional measured values can be generated in this way.

These additional measured values of a positive control make it possible to evaluate the test strips in the wet state. The nanomaterials according to the invention as luminescent substances and in particular corresponding nanocrystals are able to provide sufficient or sufficiently strong and clear signals in the wet state. Accordingly, the waiting times for drying the strips are advantageously eliminated, which means that the results can be generated much faster than with the currently available POCT methods.

Apart from POCT, an advantageous embodiment of the method according to the invention can also be used in a Western blot analysis for the purpose of quantifying the target molecules or the analyte. In general, an embodiment of the method according to the invention in which the rapid test is a lateral flow assay or a vertical flow assay or Western blot is preferred. The target proteins on the membrane in the Western blot method are labeled with an antibody or IgG, for example. The immobilized antibodies or IgGs are then made visible to the naked eye with the chemicals or protein A-coupled nanomaterials using a camera. Visualization and semi-quantitative evaluation are carried out, for example, using appropriate chemicals and the chemiluminescence technique.

In a further development of the method, ligands such as the Haevy chain of IgGs and/or streptavidin are transferred to a substrate such as an SDS gel as reference proteins. After immunodetection, the membranes are preferably treated with ligand-bound nanomaterials. The nanomaterials bind to specific IgGs that have recognized their targets, together with one or both reference proteins of known concentration. Thus, the unknown concentrations can be determined from the signals of the standard series and the reference protein of known concentration (see FIG. 7).

A further important aspect of the invention relates to the use of big data approaches and/or artificial intelligence to enable improved individualized dosing of therapeutics. In particular, this preferably relates to the creation of a corresponding database. It is also particularly preferred that quantitative data from the method according to the invention is used for this purpose.

Currently, humans and animals are treated with the medications based on existing protocols and/or characteristics, such as age, weight and gender. By means of the present invention, the degree of individual metabolization can be determined “on site”. In humans (and animals) of the same sex, age and weight, body functions such as kidney and liver function may be different. Currently, there are no quantitative POCT kits that can solve these tasks and enable TDM. However, the digitization of data for the treatment of humans and animals offers numerous advantages. When securing personal data, information such as age, gender, origin, previous illnesses and vital functions can be stored anonymously in the form of a barcode (see FIG. 8A). As an example, only the most important indicators and tests have been entered in FIG. 8A.

HPLC and ELISA can be used as the gold standard for determining the measuring ranges and defining the interfering factors. Parallel measurements, such as HPLC and ELISA, are an indispensable prerequisite for the certification of quantitative POCT products, as shown in FIG. 8B. In the medium term, corresponding databases with such data can be used to confirm the therapeutic areas, for example for cytostatics and antibiotics. This should generate data that can be used to make more precise dose adjustments for the substances. By analyzing the stored or historical data, a better dose recommendation for the substances should be possible. It should be noted that the decision remains with the treating physician. By storing the data, algorithms from the field of data analytics and machine learning can also be used.

By storing the data, big data approaches can also be pursued. For the purpose of individual dosing of the medication, certification of the POCT products and creation of the big data or database, individual TDM examinations can be carried out for each person (see FIG. 8B). These TDM examinations are carried out using the method according to the invention and/or using gold standard methods. These measurements can be performed before, during and/or after medical treatments for the purpose of individualized dosage recommendations. The resulting dosages can also be stored in the form of a barcode for the respective patient.

With the help of population pharmacokinetic analyses, digitalization, use of the data obtained and/or artificial intelligence, the possible influencing factors on the kinetics can be identified and quantified. The larger and more comprehensive the personal data collected, the smaller the deviations in medication dosage.

Previous data shows that real measurements are only necessary to update, maintain and expand the digital data. Ideally, artificial intelligence (AI) in the form of software or an app based on the stored personalized data (see FIG. 8A) can enable individualized dosage recommendations without TDM measurements (see FIG. 8B). The stored data and the clinical knowledge gained from it preferably serve as a memory for the artificial intelligence. The artificial intelligence can recommend an exact dosage to the physicians based on the stored information.

1. Method for the absolute quantification of at least one analyte by a rapid test including spectroscopic determination, in which
at least one nanomaterial is used as a luminescent substance for quantification, which interacts directly or indirectly with the analyte, wherein
at least one signal generated by the nanomaterial is measured, and wherein
an internal and/or external standard series is also measured and subsequently
the generated signal is standardized or referenced using the internal and/or external standard series, so that an absolute quantification of the analyte is achieved.

2. Method for the absolute quantification of at least one analyte according to claim 1, wherein a background material, a component, a plastic part, a plastic housing and/or a nitrocellulose membrane of the rapid test is used as the target to be measured for measuring an internal standard series, by which a constant measurement signal can be measured for defined measurement parameters independently of the concentration of the analyte and thus this background measurement signal can be used for standardization of the generated signal of the nanomaterial.

3. Method for the absolute quantification of at least one analyte according to claim 1, wherein a control line or a control position of the rapid test is measured to measure an internal standard series and the measured value obtained is used as a reference value for standardization of the generated signal of the nanomaterial, with a supplementary calculation of the analyte using a 4-parameter logistic.

4. Method for the absolute quantification of at least one analyte according to claim 1, wherein the measurement of the internal and/or the external standard series is carried out using Raman-labelled targets and/or fluorescence-labelled targets.

5. Method for the absolute quantification of at least one analyte according to claim 1, wherein, in order to measure an internal standard series, the at least one target is measured with different intensities of an irradiated light, with different laser powers and/or with a changed laser focus, so that a plurality of measurements of a standard series are obtained.

6. Method for the absolute quantification of at least one analyte according to claim 1, wherein for a measurement of an external standard series a plurality of measuring surfaces are provided, each with a different concentration of a separately prepared signal-active material and/or at least one nanomaterial, the signal-active material and/or the nanomaterial being immobilized on an attachment or on the rapid test and optionally sealed.

7. Method for the absolute quantification of at least one analyte according to claim 1, wherein at least one nanomaterial as luminescent substance is an IgG-coupled Raman-active nanomaterial, and/or the measurement of the signal generated by the nanomaterial is carried out by surface-enhanced Raman scattering, so that the generated signal is a surface-enhanced Raman scattering signal.

8. Method for the absolute quantification of at least one analyte according to claim 1, wherein the rapid test is a lateral flow assay or a vertical flow assay or Western blot and/or quantum dots upconversion and/or nanomaterials are used for the quantification of the analyte, the nanomaterials being coated with a self-assembling single layer or a monolayer of organic molecules as Raman markers and/or such materials or nanomaterials being coupled to ligands, such as protein A, protein G, protein A/G, protein L, biotinylated antibodies or enzyme-coupled and fluorescent antibodies, for the quantification of the analyte.

9. Method for the absolute quantification of at least one analyte according to claim 1, wherein the at least one nanomaterial is a nanocrystalline luminescent substance which comprises LaPO4:Ce3+, LaPO4:Pr3+, LuPO4:Pr3+, LaPO4:Tm3+LuPO4:Dy3+, LuPO4:EU3+, LuPO4:Tb3+, LuPO4:Tm3+ and is formed therefrom and/or that the at least one nanomaterial is a nanomaterial or microparticles produced by doping from a plastic polymer.

10. Method for the absolute quantification of at least one analyte according to claim 1, wherein the measurement is carried out by a UV, UV-VIS, IR, fluorescence and/or Raman spectrometer.

11. Method for the absolute quantification of at least one analyte according to claim 1, wherein a measurement and in particular a spectroscopic examination of a positive and a negative control is carried out in parallel with the measurement of the signal of the nanocrystalline luminescent substance generated by the nanomaterial.

12. Method for the absolute quantification of at least one analyte according to claim 1, wherein at least one nanocrystalline luminescent substance is used for measuring the standard series and/or at least one fluorescent dye is used as luminescent substance for quantifying the analyte.

13. Method for the absolute quantification of at least one analyte according to claim 1, characterized by a simultaneous or immediately successive exposure of the standard series and the at least one nanomaterial by spectroscopy in a spectrum range from UV to IR.

14. Device for the absolute quantification of at least one analyte by a rapid test that uses surface-enhanced Raman scattering, with
at least one nanomaterial as a luminescent substance for quantification, which interacts directly or indirectly with the analyte, the rapid test being intended to be measured by a spectrometer or by at least one filter and at least one sensor in order to measure at least one signal generated by the nanomaterial and an internal and/or external standard series, Raman-labeled targets and/or fluorescence-labeled targets, wherein
using a data processing device, the concentration of the analyte is standardized or referenced using the internal and/or external standard series, so that an absolute quantification of the analyte is achieved.

15. Use of the method according to claim 1 in human medicine, in veterinary medicine and/or in the food industry, in order to carry out a quantitative determination of active substances, medicaments and/or toxins by a rapid test.

16. Method for creating a database for improved individual dosing of therapeutics, wherein absolutely quantified measured values and/or further measured data and/or parameters of the patient obtained by means of the method according to claim 1 are used to adjust or personalize the medication dose and/or to make dosing recommendations for medications or active ingredients for future patients by means of AI algorithms and/or by big data approaches.
